(19) United States
(12) Patent Application Publication
(10) Pub. No.: US 2006/0116537 A1
(43) Pub. Date: Jun. 1, 2006

(54) Aluminium fluoride

(75) Inventor: Paolo Cuzzato, Ponzano Veneto (IT)

Correspondence Address: CONNOLLY BOVE LODGE & HUTZ, LLP, P O BOX 2207, WILMINGTON, DE, 19899, US

(73) Assignee: Solvay Solexis S.P.A., Mailand (IT)

(21) Appl. No.: 10/541,142

(22) PCT Filed: Dec. 23, 2003
(86) PCT No.: PCT/EP03/14869

§ 371 Date: Jul. 27, 2005

(30) Foreign Application Priority Data
Jan. 3, 2003 (IT) MI2003A000006
Jan. 3, 2003 (IT) MI2003A000007

(51) Int. Cl. C01F 7/50; B01J 27/132; B01J 27/125; C07C 19/08

(52) U.S. Cl. 570/166; 423/489; 502/231; 502/228

(57) ABSTRACT

Aluminium fluoride obtainable by impregnation of aluminium fluoride having a fluorine content not less than 90% with an aqueous solution containing one or more alkali metal salts.

The present invention relates to a aluminium fluoride having improved activity and the related process of preparation.

It is well known that aluminium fluoride (AlF₃) is an inorganic solid that is widely used in the chemical industry as a heterogeneous catalyst, for example in reactions of the Friedel-Crafts type or in reactions of halogen exchange or of addition of hydrogen halides on multiple carbon-carbon bonds, on account of its high-Lewis acidity. See, for example, patent FR 1,383,927, where AlF₃ is used for catalysing the addition of HF to acetylene. AlF₃ is also used as a support for other catalytic phases.

It is also known that the catalyst AlF₃, see for example the aforementioned patent FR 1,383,927, gives rise to parasitic reactions that lead to the formation of unwanted by-products, or to the formation of carbon residues that adhere to the catalyst and reduce its activity. This last-mentioned phenomenon is well known to a person skilled in the art of heterogeneous catalysis, as fouling, or more specifically coling when the contaminants consist of a coal-like substance. The formation of by-products and the phenomenon of “fouling” have an adverse effect on industrial processes, owing to the higher consumption of raw materials and to the down-times that are necessary for regenerating the catalyst.

There was a perceived need for an improved catalyst that does not have the aforementioned shortcomings.

The Applicant has found, surprisingly and unexpectedly, that this technical problem can be solved aluminium fluoride obtainable by treating, for example by impregnation, a starting aluminium fluoride having a fluorine content not less than 90%, preferably not less than 95% relative to the stoichiometric, with alkali metal derivatives, preferably with an aqueous solution containing one or more alkali metal salts.

The aluminium fluoride to be treated can be any aluminium fluoride known in the art, having, as stated, a fluorine content not less than 90%, preferably not less than 95% relative to the stoichiometric, with a surface area not less than 25 m²/g, and consists preferably substantially of the crystalline phase designated γ (gamma).

A preferred starting aluminium fluoride is a crystalline solid that can generally be obtained by fluorination of alumina with HF. Methods of synthesis of AlF₃ from alumina are known in the art, see for example U.S. Pat. No. 6,187,280 and U.S. Pat. No. 6,436,362 in the name of the Applicant.

Fluorination of alumina can be effected with HF at a temperature from 250° C. to about 450° C., preferably above 300° C. and up to 400° C., for a sufficient time to obtain a quantity of fluorine not less than 90%, preferably not less than 95%.

It is advantageous for the HF partial pressure to be low, especially at the beginning of fluorination, so as to moderate the development of heat that could cause a local increase in temperature above the limits stated above.

It is possible to dilute HF with a gas that is inert under the conditions of fluorination, for example air or nitrogen. The HF partial pressure is generally between 0.1 and 0.5.

Better temperature control can be achieved by carrying out the reaction in a fluidized bed, and this is the preferred way of effecting the fluorination. In this case the alumina to be fluorinated has a granulometry compatible with application in a fluidized bed.

In general the alumina used contains less than 0.1 wt. % of each of the contaminants that are undesirable in the final product AlF₃, for example iron and sulphates:

If the aluminas are in hydrated form, it is preferable for fluorination to be preceded by a calcining stage in air or nitrogen, at a temperature between 300° C. and 400° C. This restricts the evolution of water during the reaction, which is undesirable as it promotes corrosion of the equipment.

The aluminium fluoride thus obtained preferably has the characteristics. described in the cited patents; in particular its surface area is generally not less than 25 m²/g, and it consists normally principally of the crystalline phase designated γ (gamma).

By alumina we mean an oxide of aluminium, possibly hydrated, preferably in the crystalline form of boehmite or pseudo-boehmite, optionally containing silicon dioxide (silica), generally in a quantity up to 15 wt. %. This last-mentioned composition is commonly called silico-aluminas In the present patent application the term “alumina” is used to include silico-aluminas. The physical form of these aluminas can be as powder or as granules (pellets).

The aluminas containing silica can be prepared by methods known in the prior art, for example by spraying of suitable precursors. Such aluminas are commercially available products, for example with the trademarks Pural® and Siral® of the company Sasol.

Aluminas, aluminas containing silica, and aluminium fluorides are characterized by techniques that are well known to a person skilled in the. art of the characterization of solids: the surface area (SA) is measured by adsorption of nitrogen according to the BET method; pore volume is measured by mercury intrusion at high pressure; the crystalline phases are determined by X-ray diffraction; composition analyses are carried out by wet methods according to known methods, or by X-ray fluorescence by comparing with standards prepared on the same matrix using calibrated additions.

The alkali metals of the salts used for treating the starting aluminium fluoride are those belonging to Group I of the periodic table; preferably sodium and potassium or their mixtures, and more preferably potassium, are used as alkali metals.

The aqueous solution which can be used in the treatment contains one or more salts of the said metals.

Preferably, in the alkali metal salts used, the anion is derived from an inorganic acid, preferably a strong acid, for example hydrochloric acid or nitric acid.

The concentration of the said salts in the solution, in particular in the aqueous solution (or in water) varies from 0.1 M to 0.0001 M, preferably from 0.01 M to 0.001 M. More concentrated solutions can have an inhibitory effect on the activity of the catalyst as well as on the parasitic reactions.

A further object of the present invention is a process for preparing the aluminium fluoride catalyst described above, by treating a starting aluminium fluoride, having a fluorine content not less than 90%, preferably not less than 95% relative to the stoichiometric, for example by impregnation, with an alkali metal compound, preferably with an aqueous solution containing one or more alkali metal salts.

The alkali metal salts and the concentrations of the corresponding aqueous solutions are as stated above.

Contact between the starting AlF₃ and the impregnating solution can be effected in any technically realizable manner. At the laboratory scale it is generally simplest to immerse the particles of solid in the solution. On an industrial scale the liquid can for example percolate through or can be sprayed onto a bed of solid; these methods are well known to a person skilled in the art of preparation of catalysts.

When the treatment is carried out by impregnation by immersing the particles of the solid in the aqueous solution, stirring is preferred.

The temperature and the pressure at which contact occurs are preferably chosen so as to maintain the solution in the liquid state; it is possible, for example, to work at atmospheric pressure and room temperature.

The treatment time varies according to the method selected for treating the AlF₃; generally, for example in the case of dispersion in solution, the contact time is at least 30 minutes. The Applicant has found that times of at most 2 hours are industrially particularly useful.

After treatment with the aqueous solution of alkali metals, the aluminium fluoride is preferably treated to remove optional excess solution, for example by filtration, followed by dehydration at temperatures between 100° C. and 150° C., preferably 100-130° C.

After dehydration, the solid is preferably calcined in a stream of inert gas, for example nitrogen or helium, at a temperature generally from 300° C. to 450° C., preferably from 350° C. to 400° C.

It has been found that it is important that calcining should preferably take place at a temperature not below 300° C. and not above 450° C. In fact, below 300° C. elimination of the adsorbed water can be undesirably slow, and above 450° C. there can be partial degradation of the solid.

If the aluminium fluoride is to be used in a fluidized bed reactor, the starting alumina should preferably have a granulometry that is compatible with the said use, as is known to a person skilled in the art.

The Applicant found, surprisingly and unexpectedly, that the aluminium fluoride according to the invention can be advantageously used as catalyst and the secondary reactions are reduced, therefore the selectivity of processes is improved relative to that obtained using AlF3 as such, not treated with alkali metal.

The processes in which the aluminium fluoride according to the invention can be used are for example reactions of the Friedel-Crafts type or reactions of halogen exchange or of addition of hydrogen halides on multiple carbon-carbon bonds, on account of its high Lewis acidity. In particular, the aluminium fluoride can be used in the isomerization of HCFCs, for example the isomerization of 123a to 123.

For the industrial applications of HCFC 123 it is important to obtain the compound free from 123a. In the presence of 123a there may be formation of HCl, which corrodes the metallic parts, in particular circuits, of industrial plants.

The reaction of isomerization takes generally place in the gas phase at a temperature from 180° C. to 400° C., preferably from 220° C. to 320° C.

The contact times with the aluminium fluoride in this reaction are generally between 5 and 100 seconds.

The AlF₃ according to the present invention can also be used as support for preparing other catalysts.

The present invention relates also to catalysts, in particular chromium catalysts, comprising in particular chromium compounds, supported on the AlF₃ according to the invention. In particular, the chromium catalysts are suitable for use in the heterogeneous phase in reactions of gas-phase halogen exchange on halogenated organic compounds.

Other catalysts according to the invention comprise for example other transition metal derivatives, such as derivatives based on group VIII metals.

It is known that heterogeneous gas-solid phase catalysis is widely used in the chemical industry in various synthesis processes relating to halogenated organic compounds. We may mention for example the fluorination of chlorinated compounds with anhydrous HF, the disproportionation of chlorofluorinated compounds, addition of BF to halogenated olefins, etc.

The catalyst most used in these reactions is based on compounds of Cr(III), for example oxides or oxyhalides, as they are, or supported. We may mention for example U.S. Pat. No. 4,967,023 and U.S. Pat. No. 5,345,014 in the name of the Applicant.

It is known that in fluidized bed processes it is necessary to use a supported catalyst, because the mechanical properties of chromium compounds, for example mechanical strength, are not adequate for this use. The support must be available in a physical form suitable for this application and must possess good properties of mechanical strength and of chemical resistance in the reaction environment. This last requirement is what limits the choice of support, which in nearly all the fluidized bed catalysts consists of fluorinated alumina/aluminium fluoride. By fluorinated alumina we mean a compound containing a quantity of fluorine less than the stoichiometric corresponding to AlF₃.

Catalysts based on aluminium fluoride, or on fluorinated alumina, when they come into contact with organic molecules, generally also give parasitic reactions. that lead to the formation of unwanted by-products, or promote the formation of carbon residues, which adhere to the catalyst and reduce its activity. There was a perceived need for catalysts for use in a fluidized bed in the reactions of gas-phase halogen exchange on halogenated organic compounds, which would make it possible to reduce the quantity of by-products, thus increasing selectivity for the main product.

Now the Applicant found, surprisingly and unexpectedly, that this technical problem can be solved with the AlF₃ supported catalyst according to the invention.

One particular object of the present invention is a catalytic composition comprising trivalent chromium supported on the aluminium fluoride according to the invention.

The content of chromium in the catalyst is generally 1-20 wt. %, preferably 5-15 wt. %.

The catalyst can be used in a fluidized bed reactor, in particular if it has a compatible granulomietry for this use. Such catalyst can be obtained when the support presents a suitable granulometry as described above.

A further object of the present invention is a process for preparing the catalyst according to the invention which comprises treatment of the aluminium fluoride according to the invention with a solution comprising at least one catalytically active metal, preferably with a solution of a chromium compound and more preferably with an aqueous solution of a water-soluble salt of Cr(III).

The treatment of the AlF₃ according to the invention with catalytically active metals, in particular chromium salts can be effected by conventional methods.

It is preferable to use the method called “dry impregnation”, known in the prior art by the English term “incipient wetness impregnation”. See, for example, U.S. Pat. Nos. 4,967,023 and 5,463,151 in the name of the Applicant.

Dry impregnation is carried out by impregnating a determined quantity of AlF₃ according to the invention, obtained from treatment with the solution of alkali metal salts, with a concentrated solution of a water-soluble derivative of the catalytically active metal, in particular a salt of Cr(III), for example Cr(III) chloride. The volume of the impregnating solution is preferably less than or equal to the volume of the pores of the support, in order to avoid adhesion between the granules of the solid.

After treatment with chromium salts, drying is generally carried out, preferably at moderate temperature, for example at 120° C., to evaporate the water and deposit the salts on the support. If necessary, the stage of impregnation+drying is repeated several times until the required quantity of chromium is obtained on the support.

After the last drying operation the catalyst can be transferred for example to a tubular reactor and calcined for some hours at temperatures from 300° C. to 400° C., preferably from 350° C. to 400° C., in a stream of inert gas, for example nitrogen. Final activation is preferably carried out with a fluorinating agent, working at a temperature in the range stated above. Generally, anhydrous HF flows into the same reactor, gradually reducing the flow of inert gas until the gas mixture hag the required HF concentration. It is also possible to use pure HF. Alternatively, the catalyst can be transferred to the fluorination reactor and activated in situ with the same mixture of reagents+HF.

The Applicant found that the order of the aforementioned treatments has an influence on the activity of the catalyst.

Consequently a particular embodiment relates to a process for manufacturing the catalyst according to the invention comprising the manufacture of the aluminium fluoride according to the invention following the process described above.

It is preferred to carry out the treatment of the aluminium fluoride with the solution comprising a catalytically active metal after treating AlF₃ with the solution of alkali metal salts.

The catalysts according to the invention, in particular those containing chromium salts, can be used in various reactions of gas-phase halogen exchange on halogenated organic compounds, for example those in which compounds fluorinated in the gas phase are used.

We may mention, for example, the reaction of disproportionation of hydrochlorofluorocarbons (HCFCs), for example of HCFC-123 to HCFC-122 and HCFC-124, according to the following scheme:
2CF₃—CHCl₂→CF₃—CHClF+CF₂Cl—CHCl₂
in parallel with this reaction there is also formation of the chlorofluorocarbon (CFC) 113a (CF₃—CCl₃) according to the following side reaction:
2CF₃—CHCl₂→CF₃—CH₂Cl+CF₃—CCl₃

It is known that the CFCs, such as CF₃—CCl₃, are compounds that are suspected to be harmful to the ozone layer, and their production and marketing have been banned by international regulations.

It was found, unexpectedly and surprisingly, that the catalysts according to the present invention are more selective than those known in the prior art, and in the case of the aforementioned reaction of disproportionation of HCFC-123 the quantity of CFC 113a that forms is lower, at equal conversion of the main product, compared to that obtainable with the catalysts according to the prior art.

In general the catalysts of the present invention are used in processes of synthesis involving halogenated organic compounds. We may mention for example the fluorination of (H)CFC compounds with anhydrous HF, for example chlorinated compounds, the disproportionation of (H)CFC compounds, for example chlorofluorinated compounds, addition of HF to halogenated olefins, etc.

Some examples are given below, which illustrate but do not limit the present invention.

### EXAMPLES

### Example 1a

### Preparation of the Catalyst

A 12-g sample of aluminium fluoride, in the form of fine powder suitable for use in fluidized beds (obtained by fluorination of a silico-alumina of the type Sasol Siral® 1.5, as described in U.S. Pat. No. 6,187,280), was suspended for 60 minutes, with continuous stirring, at room temperature (20-25° C.), in 400 cm³ of an aqueous solution containing KCl at a concentration of 0.001 mol.

Next, the sample was filtered to remove the excess solution, then it was dried in a stove at 105° C. for 4 hours and finally loaded in an electrically-heated tubular reactor, and calcined at 380° C. for 4 hours in a stream of helium.

### Example 1b

### Preparation of the Catalyst

The same procedure as in example 1a was repeated but using a KCl solution at a concentration of 0.01 mol/L. Example 1c

### Preparation of the Catalyst

The same procedure as in example 1 a was repeated but using a KCl solution at a concentration of 0.1 mol/L.

### Example 2a

Isomerization of HCFC-123a to HCFC-123

10 g (approx. 11 cm³) of the AlF3 of example 1a was loaded in a tubular reactor with an inside diameter of approx. 20 mm, with electrical resistance heating. It was raised to a temperature of 380° C. in a helium stream and was maintained at this temperature for 4 hours in order to eliminate any traces of absorbed moisture.

Then the temperature was adjusted to the reaction temperature of 350° C. and the reactor was supplied with a mixture of isomers of HCFC-123 (C₂F₃HCl₂) containing, as percentage by weight, 11% of isomer 123a (CF₂Cl—CHFCl), 89% of isomer 123 (CF₃—CHC12J and traces (approx. 0.05%/o) of CFC-113, the mixture having been diluted in helium in the volume ratio of 30% initial mixture/70% helium.

The pressure in the reactor was atmospheric pressure.

The total feed flow rate was 400 s-cm³/h (standard cm³ measured at atmospheric pressure and at a temperature of 25° C.).

The nominal contact time at the reaction temperature and at atmospheric pressure was equal to 48 s.

The gases leaving the reactor were sampled in the gas phase and analysed by GLC.

The activity of the catalyst remains constant throughout the experiment.

The following analysis was representative of the results obtained. The percentages were percentages by weight.

| | |
|---|---|
| 123 | 97.1% |
| 123a | 0.3% |
| others | 2.6% |

“Others” means various halogenated compounds with 2 carbon atoms, mainly HCFC-124 (CF₃—CHFCl), CFC-113a (CF₃—CCl₃), and CFC-1111 (CCl₂═CFCl).

Conversion of 123a to 123 was 97%, and the selectivity of the reaction was approx. 97.5%.

### Example 2b

The reactor in example 2a was loaded with 10 g of the catalyst prepared as in example 1b, which was treated as in example 2a.

A mixture of 123/123a (98.5/1.5%), diluted in helium in a volume ratio of 30% initial mixture/70% helium, was supplied at 350° C. The total flow rate was 550 cm³/min.

The following analysis is representative of the results obtained. The following percentages are by weight.

| | |
|---|---|
| 123 | 99.0% |
| 123a | 0.3% |
| Others | 0.7% |

The isomeric composition of the 123 in the products was the same as in the preceding example.

The selectivity was greater than 99%.

### Example 2c

The reactor in the preceding example was loaded with 10 g of a catalyst prepared as in example 1c, which was treated as in the previous examples.

A mixture of 123/123a (isomeric composition 89.1%-10.9%), diluted in helium in the same volume ratio as in the previous examples, was supplied at a temperature of 350° C., with flow rate and contact time the same as in example 2a.

The following analysis was representative of the results obtained. The percentages are percentages by weight.

| | |
|---|---|
| 123 | 94.0% |
| 123a | 5.3% |
| Others | 0.7% |

The selectivity was the same as in example 2b.

### Example 3 (Comparative)

The same AlF₃ used as starting material in the previous examples was used in the reaction of isomerization as such, without impregnation with the solution of alkali metal salt

10 g was loaded into the reactor described above and treated as in the previous examples; at a temperature of 350° C., the reactor was supplied with a mixture of HCFC-123/123a, containing 12% of isomer 123a, diluted in helium in the same volume ratio as used in the previous examples.

The total flow rate was 570 s-cm³/h. The nominal contact time was 33 secs. The gases were sampled and analysed as in the previous examples; the following analysis was representative of the results obtained (wt. %):

| | |
|---|---|
| 123 | 89.0% |
| 123a | 0.3% |
| Others | 10.7% |

In this experiment the selectivity was much lower (<90%) than was obtained in the same reaction using the aluminium fluoride of the invention.

### Example 4-a

### Preparation of the Support for Chromium Catalyst

An 8-g sample of aluminium fluoride, in the form of fine powder suitable for use in fluidized beds (obtained by fluorination of a silico-alumina of the type Sasol Siral® 1.5, as described in example 2 of U.S. Pat. No. 6,187,280), was suspended for 60 minutes, stirring continuously, at room temperature (20-25° C.), in 800 cm³ of an aqueous solution containing KCl at a concentration of 0.001 mol.

Next, the sample was filtered to remove the excess solution, then it was dried in a stove at 120° C. for 4 hours and was finally loaded into an electrically-heated tubular reactor, and calcined at 360° C. for 2 hours in a stream of helium.

### Example 4-b

### Preparation of the Support

The same procedure as in example 4a was followed, but using a solution of KCl at a concentration of 0.12 mol/L.

### Example 4-c

### Preparation of the Support

The same procedure as in example 4a was followed, but using a solution of NaCl at a concentration of 0.001 mol/L.

### Example 4-d

### Preparation of the Support

The same procedure as in example 4a was followed, but using a solution of NaCl at a concentration of 0.12 mol/L.

### Example 4-e

### Preparation of the Support

The same procedure as in example 4a was followed, but using a solution of KNO₃ at a concentration of 0.12 mol/L.

### Examples 5a-5e

### Preparation of Catalysts According to the Invention

Each of the supports obtained in examples 4a-4e was treated in the following way: 4 g of support was impregnated by “dry impregnation” with 4 g (approx. 3 cm³) of an aqueous solution of CrCl₃ 50 wt %, divided into five portions. The nominal content of chromium on the catalyst obtained was approx. 8 wt. %.

After impregnation. the samples were first dried at 90° C. in a muffle furnace, then transferred to a tubular reactor and treated at 360° C. in a stream of helium. They were then treated, at the said temperature, with a helium/HF mixture for 6 hours to complete the activation.

### Example 5f (Comparative)

An 8-g sample of aluminium fluoride, in the form of fine powder suitable for use in fluidized beds (obtained by fluorination of a silico-alumina of the type Sasol Siral® 1.5, as described in example 2 of USP 6,187,280), was treated with CrCl₃ as described for examples 2a-2e. The final chromium content was approx. 8wt. %.

### Example 6a

### Disproportionation of HCFC-123 to HCFC-122 and HCFC-124

1.5 g of the catalyst prepared according to example 5a was loaded into a tubular reactor with diameter of about 8 mm, having a porous diaphragm at the base, and heated electrically.

The reactor was heated to 360° C. in a stream of helium. At this temperature, the catalyst was fluorinated with anhydrous HF (5 g HF in total) for 1.5 hours for the purpose of eliminating the absorbed moisture.

The temperature inside the reactor was then adjusted to 280° C., maintaining a stream of helium, and supply of He/HCFC-123 mixture, consisting of 9.4 cm³/min of helium and 3.4 cm³/min of HCFC-123, was started.

The products leaving the reactor were sampled in the gas phase and were analysed by gas chromatography. Sampling was effected after about 2 h of operation, so that the reactor was in operation under the specified conditions.

The following analysis is representative of the results obtained. The percentages are wt. %.

| | | |
|---|---|---|
| HCFC-123 | (isomers of C₂HF₃Cl₂) | 84.78 |
| HCFC-124 | (isomers of C₂HF₄Cl) | 10.19 |
| HFC-125 | (C₂HF₅) | 0.88 |
| CFC-114a | (CF₃CFCl₂) | 0.20 |
| CFC-113 | (isomers of C₂F₃Cl₃) | 0.10 |
| CFC-116 | (C₂F₆) | 0.01 |
| CFC-112 | (isomers of C₂F₂CCl₄) | 0.01 |
| CFC-115 | | below range |
| others | | complement to 100 |

The conversion of HCFC-123 was 15.2%, the summation of all the CFCs (Σ_{CFC}) was 0.32% and the selectivity for CFC was 2.1%.

The results were also shown in Table 1.

### Examples 6b-6e

The reaction of disproportionation of HCFC-123 was repeated in the same conditions as in example 6a, using the catalysts of examples 5b-5e respectively.

The results are shown in Table 1.

### Example 7 (Comparative)

The reaction of disproportionation of HCFC-123 was carried out in the same conditions as in the previous examples but using the catalyst prepared in example 5f. The results obtained were shown in Table 1.

Comments on Table 1

Table 1 shows that with the catalyst according to the present invention the selectivity for CFCs was lower than with the catalyst of the prior art having identical composition but prepared with a support not treated with the solution of alkaline salts.

TABLE 1
Thwastable shows the conversion of HFC 123, the total quantity (ΣCFC)
and the selectivity for CFCs of the reaction of disproportionation of
HCFC-123, in relation to the composition of the salt solution used
for treating the support. The quantity of chromium in the catalysts
used wasapprox. 8 wt. %.

| | Catalyst | | Conversion | | Selectivity |
|---|---|---|---|---|---|
| Exam- | Exam- | Solution for | HCFC-123 | ΣCFC | CFC |
| ple | ple | treating AlF₃ | % | % | % |
| 6a | 5a | 0.001 M KCl | 15.2 | 0.32 | 2.1 |
| 6b | 5b | 0.12 M KCl | 23.9 | 0.45 | 1.88 |
| 6c | 5c | 0.001 M NaCl | 35.5 | 0.43 | 1.21 |
| 6d | 5d | 0.12 M NaCl | 36.3 | 0.20 | 0.55 |
| 6e | 5e | 0.12 M KNO₃ | 47 | 0.05 | 1.06 |
| 7 | 5f | — | 17.4 | 0.44 | 2.53 |

1. Aluminum fluoride obtainable by treating a starting aluminum fluoride having a fluorine content not less than 90%, relative to the stoichiometric, with an alkali metal derivative.

2. Aluminum fluoride according to claim 1, in which the surface area of the starting aluminum fluoride is not less than 25 m²/g, and which starting aluminum fluoride consists substantially of the crystalline phase designated γ (gamma).

3. Aluminum fluoride according to claim 1, in which the alkali metals of the salts that are used belong to Group I of the periodic table.

4. Aluminum fluoride according to claim 3, in which the anion of the salt is derived from an inorganic acid.

5. Aluminum fluoride according to claim 1, in which the concentration of the alkali metal salts in the aqueous solution is from 0.1 M to 0.0001 M.

6. Process for preparing the aluminum fluoride according to claim 1, in which a starting aluminum fluoride, having a fluorine content not less than 90% relative to the stoichiometric, is treated with an alkali metal derivative.

7. Process for preparing the aluminum fluoride according to claim 6, in which the starting aluminum fluoride is treated by impregnation with an aqueous solution containing one or more alkali metal salts.

8. Process according to claim 6, in which AIF₃, after impregnation, is treated to remove the excess solution, dehydrated at temperatures between 100° C. and 150° C., and calcined in a stream of inert gas, at a temperature generally from 300° C. to 450° C.

9-11. (canceled)

12. Catalyst comprising the aluminum fluoride according to claim 1 as a support.

13. Catalyst according to claim 12, comprising a chromium compound supported on the support.

14. Catalyst according to claim 13, comprising trivalent chromium.

15. Catalyst according to claim 12, comprising chromium and having a chromium content of 1-20 wt. %.

16. Process for preparing a catalyst comprising the aluminum fluoride according to claim 1 as support, comprising the treatment of the aluminum fluoride according to claim 1 with a solution comprising a catalytically active metal.

17. The process according to claim 16, wherein the solution is a solution of a chromium compound.

18. Process according to claim 17, wherein the chromium compound is a water-soluble chromium (III) salt.

19. Process according claim 16, in which the treatment of AIF₃ with the solution comprising the catalytically active metal is carried out with a volume of a concentrated aqueous solution of a water-soluble derivative of the catalytically active metal less than or equal to the volume of the pores in the support.

20. Process according to claim 16, in which the catalyst, after the treatment with the solution comprising the catalytically active metal, is dried and optionally the stage of impregnation and drying is repeated until the required quantity of metal is obtained in the catalyst.

21. Process according to claim 16, in which the catalyst is calcined at temperatures from 300° C. to 400° C., in a stream of inert gas, then activated with anhydrous HF, gradually reducing the flow of inert gas until there is the desired concentration of HF in the gas mixture.

22. Process according to claim 16, comprising the manufacture of aluminum fluoride according to a process for preparing the aluminum-fluoride according to claim 1, in which a starting aluminum-fluoride, having a fluorine content not less than 90% relative to the stoichiometric, is treated with an alkali metal derivative.

23. Process according to claim 22, wherein the treatment of the aluminum fluoride with the solution comprising a catalytically active metal is carried out after treating AlF₃ with the solution of alkali metal salts.

24. Reactions of gas-phase halogen exchange on halogenated organic compounds, fluorination of (H)CFC compounds or addition of HF to halogenated olefins which comprise the catalyst as claimed in claim 12.

25. A Friedel-Crafts type, in reactions of halogen exchange and of addition of hydrogen halides on multiple carbon-carbon bonds which comprises a catalyst which comprises the aluminum fluoride as claimed in claim 1.

26. A reaction of isomerization of (HCFC-123a) to (HCFC-123) which comprises a catalyst which comprises the aluminum fluoride as claimed in claim 1.

27. Aluminum fluoride according to claim 4, in which the alkali metals of the salts that are used belong to Group I of the periodic table are potassium and said inorganic acid is a strong acid.
